(19) United States
(12) Patent Application Publication
(10) Pub. No.: US 2018/0165812 A1
(43) Pub. Date: Jun. 14, 2018

(54) METHOD FOR DETERMINING TISSUE PROPERTIES OF TUMORS

(71) Applicant: Siemens Healthcare GmbH, Erlangen (DE)

(72) Inventors: Thomas FLOHR, Uehlfeld (DE); Bernhard SCHMIDT, Fuerth (DE)

(73) Assignee: Siemens Healthcare GmbH, Erlangen (DE)

(21) Appl. No.: 15/832,875

(22) Filed: Dec. 6, 2017

(30) Foreign Application Priority Data
Dec. 12, 2016 (DE) 102016224717.4

(51) Int. Cl. G06T 7/00; A61B 6/03; A61B 6/00; G06T 11/00; G06T 7/40

(52) U.S. Cl. CPC G06T 7/0016; A61B 6/032; A61B 6/481; G06T 2207/30096; G06T 7/40; G06T 2207/10081; G06T 11/005

(57) ABSTRACT

A method includes acquiring contrast medium-enhanced projection measurement data from the examination region, including at least two spectral projection measurement data sets. Further, image data is reconstructed based upon the acquired projection measurement data, the image data including at least two spectral image data sets. Subsequently, texture parameters are determined based upon the reconstructed image data and a parameter analysis is carried out based upon the parameter database. In addition, an image analysis apparatus and a computed tomography system are described.

### PRIORITY STATEMENT

The present application hereby claims priority under 35 U.S.C. § 119 to German patent application number DE 102016224717.4 filed Dec. 12, 2016, the entire contents of which are hereby incorporated herein by reference.

### FIELD

At least one embodiment of the invention generally relates to a method for determining tissue properties in an examination region. At least one embodiment of the invention also generally relates to an image analysis apparatus. Furthermore, at least one embodiment of the invention generally relates to a computed tomography system.

### BACKGROUND

With the aid of modern imaging methods, two or three-dimensional image data is often created which can be used for visualizing an imaged examination object and also for other uses.

The imaging methods are often based upon the detection of X-ray radiation wherein so-called “projection measurement data” is generated. For example, projection measurement data can be acquired with the aid of a computed tomography (CT) system. In CT systems, a combination of an X-ray source and, mounted opposite thereto, an X-ray detector, the combination being arranged on a rotating gantry, typically revolves round a scanning space in which the object under investigation (which is identified below as a patient, but without restricting the generality) is situated. The center of rotation (also known as “isocenter”) coincides with a “system axis” z. During one or more rotations, the patient is irradiated with X-ray radiation from the X-ray source, wherein projection measurement data or X-ray projection data which describes the X-ray attenuation by the patient in this irradiation direction is detected with the aid of the X-ray detector positioned opposite thereto.

The projection measurement data, referred to as projection data for short, is dependent, in particular, on the construction of the X-ray detector. X-ray detectors typically have a plurality of detection units which are most usually arranged in the form of a regular pixel array. The detection units each generate a detection signal for X-ray radiation incident on the detection units, which signal is analyzed at particular time points with regard to intensity and spectral distribution of the X-ray radiation in order to draw conclusions regarding the examination object and to generate projection measurement data. On the basis of the projection measurement data, image data is then reconstructed. The reconstruction can be carried out, for example, with the aid of a filtered back projection.

In some types of CT imaging methods, a plurality of image recordings are carried out with X-ray radiation having different X-ray energy spectra, of one and the same examination region of a patient. This process is also denoted as multi-energy CT recording. Such a multi-energy CT recording can take place, for example, with the aid of a plurality of CT image recordings one after another or simultaneously with a plurality of X-ray sources with different X-ray voltages. Recordings can also be realized simultaneously with different energy spectra if an energy-sensitive detector is used and if, for a single CT image recording, X-ray attenuation data with different effective spectra is recorded simultaneously. This procedure can be realized, for example, with the aid of quantum-counting detectors or multi-layered detectors.

The image recordings mentioned, denoted in the following as spectral CT image recordings can be used, for example, to determine the composition of body substance or the proportions of different materials in an examination region.

In the treatment of cancers, it is often important to characterize more exactly the tumor to be treated. For example, the aggressiveness of the tumor should be determined. It is also important, before the start of a treatment, to be able to predict how a tumor will react to a particular treatment and also during the treatment, to be able to monitor the reaction of the tumor to this treatment.

Conventionally, CT imaging methods are used wherein the response of tumors to a treatment takes place with the aid of measurements of morphological variables. An example for this is the use of the RECIST criterion. In addition, technologies such as, for example, CT perfusion imaging or dual-energy CT imaging are used in order to characterize tumors, to predict their response and to monitor their treatment. However, these technologies are still in the experimental stage and are not yet established in clinical use.

Another novel approach for characterizing tumors, predicting their response to a treatment or for monitoring their response to a treatment consists in texture analysis. For texture analysis a separation filter is applied to a CT image in order to generate a series of derived images which show features for different separation scale values, for example, from fine to coarse. Typically used features are, for example, the mean value of intensity, the standard deviation, homogeneity or entropy. It has been shown that some of these features have a prediction value. For example, with the aid of a determination of the evenness of the CT image features which are separated from one another by 10 to 12 image pixels, the life expectancy of patients with liver metastases or intestinal cancer can be predicted.

Spectral CT imaging is used to calculate pseudo-monoenergetic images at different X-ray energy values, to calculate iodine images or to generate virtual non-contrast images. The iodine content in an iodine image serves to measure the local blood volume.

Through simple determination of the mean CT value in particular regions in an iodine image and a virtual non-contrast image, i.e. an image which corresponds to an image recorded without contrast medium, it was attempted to characterize tumors with regard to their benignity or malignancy, to predict their response to a treatment and to monitor their response during the treatment. Herein, a reduced CT value in the iodine image was associated with a lower iodine concentration and therefore with a successful treatment. Alternatively, the change in the CT value in lesions was also used as a function of the X-ray energy in monochromatic images for the stated treatment purposes. However, in both approaches, the additional information that is obtained through the evaluation of the CT images is restricted and the stated methods are not sufficiently reliable for clinical use.

### SUMMARY

The inventors discovered that a problem therefore exists of developing a method for determining tissue properties on the basis of CT images and a corresponding analysis device with the help of which data can be obtained on the basis of which tumors are more reliably characterizable and their response to treatments can be more precisely and reliably predicted and monitored.

At least one embodiment of the invention is directed to a device for determining tissue properties in an examination region. At least one embodiment of the invention is directed to image analysis apparatus. At least one embodiment of the invention is directed to a computed tomography system.

In at least one embodiment of the inventive method for determining tissue properties in an examination region, contrast medium-enhanced projection measurement data, which comprises at least two spectral projection measurement data sets, is acquired from the examination region. In this context, contrast medium-enhanced should be understood to mean that a contrast medium is present in the examination region during the acquisition of the projection measurement data. With the aid of the contrast medium, liquids, in particular blood can be made readily visible. Spectral projection measurement data sets should be understood in this context to mean sets of projection measurement data associated with different X-ray energy spectra. Different X-ray energy spectra should be understood to mean that the energy distribution of the X-ray radiation that contributes to the generation of the different sets of projection measurement data differs. The examination region should be understood in this context to be a sub-region of the body of a patient which is to be more closely examined with the aid of a CT imaging method. The patient should be understood in this context to be both a human to be examined and also an animal to be examined.

The image analysis apparatus according to at least one embodiment of the invention has an input interface for receiving contrast medium-enhanced projection measurement data from an examination region of a patient, the data comprising at least two spectral projection measurement data sets. The image data analysis apparatus according to at least one embodiment of the invention also comprises an image reconstruction unit for reconstructing image data on the basis of the contrast medium-enhanced projection measurement data, wherein the image data comprises at least two spectral image data sets. Part of the image analysis apparatus according to at least one embodiment of the invention is also an image analysis unit for generating a parameter database. The generation of the parameter database comprises the establishment of texture parameters in the examination region on the basis of the reconstructed image data.

Furthermore, the image analysis unit is configured to carry out a parameter analysis, preferably comprising a texture parameter analysis, on the basis of the parameter database. Advantageously, the image analysis unit is configured to determine parameter values on the basis of a plurality of spectrally different image data sets. In this way, comparative data is obtained on the basis of which additional information, for example, correlations between parameter values of different images can be obtained in order to be able to estimate the behavior of tumors more reliably. Since at least a part of the parameters of the parameter database which underlies the parameter analysis comprises texture parameters, the parameter analysis can be used for more exact and more reliable predictions and characterizations of tissue states, in particular tumors, as compared with conventional prediction methods.

The computed tomography system according to at least one embodiment of the invention has a scanning unit for acquiring projection measurement data from an examination region of a patient and an image analysis apparatus according to at least one embodiment of the invention.

A realization largely through software has the advantage that conventionally used computed tomography systems can easily be upgraded with a software update in order to operate in the manner according to at least one embodiment of the invention. In this respect, at least one embodiment is also directed to a corresponding computer program product with a computer program which is loadable directly into a storage apparatus of a computed tomography system, having program portions in order to carry out all the steps of the method according to at least one embodiment of the invention when the program is executed in the computed tomography system. Such a computer program product can comprise, apart from the computer program, additional components, if relevant, such as for example, documentation and/or additional components including hardware components, for example, hardware keys (dongles, etc.) in order to use the software.

### BRIEF DESCRIPTION OF THE DRAWINGS

The invention will now be described again in greater detail using example embodiments by reference to the accompanying drawings. In the various drawings, the same components are identified with identical reference signs. In the drawings:

FIG. 1 shows a flow diagram which illustrates a method for determining tissue properties in an examination region according to an example embodiment of the invention,

FIG. 2 shows a schematic representation of an image analysis apparatus according to an example embodiment of the invention,

FIG. 3 shows a flow diagram which illustrates a method for determining tissue properties in an examination region according to an alternative example embodiment of the invention,

FIG. 4 shows a schematic representation of a computed tomography system according to an example embodiment of the invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (procesor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

In at least one embodiment of the inventive method for determining tissue properties in an examination region, contrast medium-enhanced projection measurement data, which comprises at least two spectral projection measurement data sets, is acquired from the examination region. In this context, contrast medium-enhanced should be understood to mean that a contrast medium is present in the examination region during the acquisition of the projection measurement data. With the aid of the contrast medium, liquids, in particular blood can be made readily visible. Spectral projection measurement data sets should be understood in this context to mean sets of projection measurement data associated with different X-ray energy spectra. Different X-ray energy spectra should be understood to mean that the energy distribution of the X-ray radiation that contributes to the generation of the different sets of projection measurement data differs. The examination region should be understood in this context to be a sub-region of the body of a patient which is to be more closely examined with the aid of a CT imaging method. The patient should be understood in this context to be both a human to be examined and also an animal to be examined.

The at least two spectral projection measurement data sets can be acquired, for example, simultaneously with the aid of a dual-energy CT imaging method or another spectral CT imaging method. The two projection measurement data sets can alternatively also be acquired one after the other, the image recordings taking place contrast medium-enhanced.

Image data is then reconstructed on the basis of the acquired projection measurement data, the image data comprising at least two spectral image data sets. Spectral image data sets should be understood to mean image data sets which have been reconstructed on the basis of the aforementioned spectral projection measurement data sets. Herein, the spectral image data sets can be obtained, for example, with the aid of a base material differentiation.

Such a base material differentiation is described, for example in PHYS. MED. BIOL., 1976, Vol. 21, No. 5, 733-744, “Energy-selective Reconstructions in X-ray Computerized Tomography”, R. E. Alvarez and A. Macovski, the entire contents of which are hereby incorporated herein by reference, for the differentiation between two base materials. Herein, two projection measurement data sets or image data sets are generated, the attenuation values or density values determined for the data sets corresponding to the attenuation by the respective base materials or the concentration of the respective base materials.

The differentiation according to base materials can take place both in the projection measurement data space as well as in the image data space. In conventional uses of this technology, as typical base materials, for example iodine and water or bone and water are used, for which different scattering mechanisms, i.e. the photoelectric effect and the Compton effect, are relevant. On the basis of the base material differentiation, virtual image data sets with which different X-ray energy spectra are associated can then be calculated. I.e., virtual image data sets are calculated which correspond to image data sets that have been reconstructed on the basis of projection measurement data that was recorded with X-rays of the aforementioned X-ray energy spectra.

Subsequently, a parameter database is established in the examination region on the basis of the reconstructed image data. Finally, a parameter analysis takes place, preferably comprising a texture parameter analysis, on the basis of the parameter database.

Where it is stated in the application that parameters are determined, this should be understood to mean that corresponding parameter values are determined for particular parameters or parameter types. The expressions “parameter” and “parameter value” are thus intended to have the same meaning in this context. In this context, parameters or parameter values of the parameter database assigned to them should be understood to be image parameters, for example, the aforementioned texture parameters, but also CT mean value parameters.

CT mean value parameters should be understood to be parameters assigned to mean CT values or representing these mean CT values. The mean CT values are determined by averaging CT values in a pre-determined region. The determination of image parameters for a plurality of images to which different X-ray spectra are assigned permits, on subsequent analysis of the parameter values, these values to be compared for different spectral portions.

Herein, for example, correlations between the parameter values or the distribution of these parameter values in a plurality of images can be determined and from these correlations, conclusions can be drawn regarding the aggressiveness of tumors, their response to treatments predicted and monitored during such a treatment. The use of a contrast medium for the image recordings that are to be compared enables active regions of a tumor which are supplied with blood to be recognized. Since the database underlying the analysis also comprises texture parameters, the parameter analysis is based, according to at least one embodiment of the invention, upon a combination of spectral information and texture data, such that the analysis forms a reliable basis for a later characterization of a tumor.

According to at least one embodiment of the invention, a parameter analysis takes place at least partially on the basis of spectral image data. If the analysis takes place on the basis of the spectral image data, then the analysis can be carried out purely with blood volume images, i.e. without anatomical structures lying thereunder. For example, with exact matching, parameter values and correlations of these parameters between virtual contrast medium-free images, virtual iodine images and mixed images can be analyzed.

The image analysis apparatus according to at least one embodiment of the invention has an input interface for receiving contrast medium-enhanced projection measurement data from an examination region of a patient, the data comprising at least two spectral projection measurement data sets. The image data analysis apparatus according to at least one embodiment of the invention also comprises an image reconstruction unit for reconstructing image data on the basis of the contrast medium-enhanced projection measurement data, wherein the image data comprises at least two spectral image data sets. Part of the image analysis apparatus according to at least one embodiment of the invention is also an image analysis unit for generating a parameter database. The generation of the parameter database comprises the establishment of texture parameters in the examination region on the basis of the reconstructed image data.

Furthermore, the image analysis unit is configured to carry out a parameter analysis, preferably comprising a texture parameter analysis, on the basis of the parameter database. Advantageously, the image analysis unit is configured to determine parameter values on the basis of a plurality of spectrally different image data sets. In this way, comparative data is obtained on the basis of which additional information, for example, correlations between parameter values of different images can be obtained in order to be able to estimate the behavior of tumors more reliably. Since at least a part of the parameters of the parameter database which underlies the parameter analysis comprises texture parameters, the parameter analysis can be used for more exact and more reliable predictions and characterizations of tissue states, in particular tumors, as compared with conventional prediction methods.

The computed tomography system according to at least one embodiment of the invention has a scanning unit for acquiring projection measurement data from an examination region of a patient and an image analysis apparatus according to at least one embodiment of the invention.

Some of the essential components of the image analysis apparatus according to at least one embodiment of the invention can be configured mainly in the form of software components. This relates, in particular, to the image reconstruction unit and the image analysis unit. Fundamentally however, these components can also, in part, be realized in particular, if particularly rapid calculations are involved, in the form of software-supported hardware, for example, FPGAs or the like. Similarly, the required interfaces can be configured, for example, where only an acceptance of data from other software components is concerned, as software interfaces. However, they can also be configured as interfaces constructed from hardware, which are controlled by suitable software.

A realization largely through software has the advantage that conventionally used computed tomography systems can easily be upgraded with a software update in order to operate in the manner according to at least one embodiment of the invention. In this respect, at least one embodiment is also directed to a corresponding computer program product with a computer program which is loadable directly into a storage apparatus of a computed tomography system, having program portions in order to carry out all the steps of the method according to at least one embodiment of the invention when the program is executed in the computed tomography system. Such a computer program product can comprise, apart from the computer program, additional components, if relevant, such as for example, documentation and/or additional components including hardware components, for example, hardware keys (dongles, etc.) in order to use the software.

For transport to the computed tomography system and/or for storage at or in the computed tomography system, a computer-readable medium, for example, a memory stick, a hard disk or another transportable or firmly installed data carrier can be used on which the program portions of the computer program which are readable and executable by a computer unit of the computed tomography system are stored. For this purpose, the computer unit can have, for example, one or more cooperating microprocessors or the like.

Further particularly advantageous embodiments and developments of the invention are disclosed by the dependent claims and the following description, wherein the independent claims of one claim category can also be further developed similarly to the dependent claims or description passages of another claim category and, in particular, also individual features of different example embodiments or variants can be combined to new example embodiments or variants.

In a preferred variant of at least one embodiment of the inventive method for determining tissue properties in an examination region, the texture parameters are determined on the basis of the at least two spectral image data sets. If texture parameters are analyzed, then the texture analysis can be carried out purely with blood volume images, i.e. without anatomical structures lying thereunder. For example, with exact matching, textures and correlations between virtual contrast medium-free images, virtual iodine images and mixed images can be analyzed.

In an alternative embodiment of the method according to at least one embodiment of the invention, in the creation of the parameter database, CT mean value parameters are determined on the basis of the at least two spectral image data sets.

How predictions can be made regarding the behavior of tumors on the basis of the mean CT values is described by Miles et al. in “Colorectal Cancer: Texture Analysis of Portal Phase Hepatic CT Images as a Potential Marker of Survival”, Radiology, Vol. 250: No. 2-February 2009, the entire contents of which are hereby incorporated herein by reference.

In this variant, for example, the texture parameters can be obtained on the basis of a standard CT imaging process, so that the effort for obtaining the texture parameters is reduced. By contrast in this variant, the spectral data is used for determining the mean CT values, also named CT mean value parameters. Therefore in this variant also, the analysis takes place on the basis of spectral data and texture parameters, so that in this variant also, a combination of texture analysis and spectral analysis can be carried out, which contributes to an improved accuracy of the analysis.

In one embodiment of this variant, on the basis of the contrast medium-enhanced projection measurement data, a standard image data set is reconstructed and the texture parameters are determined on the basis of the standard image data set. Advantageously, in this variant for the texture analysis only one image data set must be investigated, which greatly reduces the effort for the complex texture analysis. A CT image data set which was created on the basis of a standard CT imaging method should be understood in this context as a standard image data set. In this method, a single projection measurement data set is created with polychromatic X-ray radiation. The standard images are reconstructed on the basis of this projection measurement data set. A differentiation according to X-ray energy values does not take place in the standard CT imaging method.

In order to obtain the standard image data set, for example, in the acquisition of the contrast medium-enhanced projection measurement data, a projection measurement data set can additionally be acquired and the standard image data set can be reconstructed on the basis of the additional projection measurement data set. The additional projection measurement data set can be obtained, for example, on the basis of a standard CT imaging process, so that the effort for obtaining the projection measurement data for the image data for the texture analysis is reduced.

Alternatively, the additional standard image data set can also be obtained as a mixed image of a plurality of spectral image data sets. In this variant, for example, spectral image data sets that are in any event needed for obtaining the mixed image are called upon so that the effort during imaging and/or the acquisition of the projection measurement data is further reduced.

In one embodiment of the inventive method for determining tissue properties in an examination region, the reconstructed image data is pseudo-monoenergetic image data, also designated pseudo-monochromatic image data. Pseudo-monoenergetic image data is typically generated on the basis of projection measurement data obtained with different X-ray energy spectra.

Pseudo-monoenergetic image data can be reconstructed, for example, on the basis of a multimaterial differentiation. Such a multimaterial differentiation or base material differentiation is described, as mentioned above, for example, in PHYS. MED. BIOL., 1976, Vol. 21, No. 5, 733-744, “Energy-selective Reconstructions in X-ray Computerized Tomography”, R. E. Alvarez and A. Macovski, the entire contents of which are hereby incorporated herein by reference, for the differentiation of two base materials.

From the data which is associated with the base materials, images associated with any desired X-ray energy spectra can be calculated. An example of this are pseudo-monoenergetic or pseudo-monochromatic images in which only a narrow frequency band of the X-ray spectrum is taken into account. For example, on use of contrast media with a method of this type, a spectral region can be restricted to a defined region in order to obtain a particularly good contrast.

Polychromatic images are determined by the recording spectrum. Virtual keV images, also known as pseudo-monoenergetic images, are secondary images which are calculated from the initial polychromatic dual-energy (high-low) images. The keV images show a strong energy-dependency in the case of materials with a high atomic number in the tissue. This different behavior should lead to different image parameters, in particular, different texture parameters. These themselves or correlations can assist in the tissue characterization.

Preferably, in at least one embodiment, the at least two spectral image data sets comprise one of the following types of image data sets:

an iodine image and a virtual non-contrast image, or

a series of monochromatic images.

Herein, monochromatic images should be understood to be the aforementioned pseudo-monochromatic images.

The different images show “other” or different information. Masking effects can thus be subtracted out. If a parameter analysis takes place on the basis of spectral image data, this can also be helpful for the determination of correlations. The aforementioned spectral image data sets can be used both for the analysis of CT mean value parameters and also for the texture analysis.

In one embodiment of the inventive method for determining tissue properties in an examination region, on the basis of the parameter analysis, one of the following items of information is determined:

a characterization of a tumor,

the expected response of a tumor to a particular treatment, or

the actual response of the tumor during a treatment.

Characterizing of a tumor should be understood in this context as meaning that the extent of the aggressiveness of a tumor is determined. Since the database forming the basis for the parameter analysis also comprises texture parameters, the texture parameters are included, in combination with spectral information, in the characterizing of the tumor. How the aforementioned information is to be determined on the basis of texture parameters is also described in detail in Miles et al.

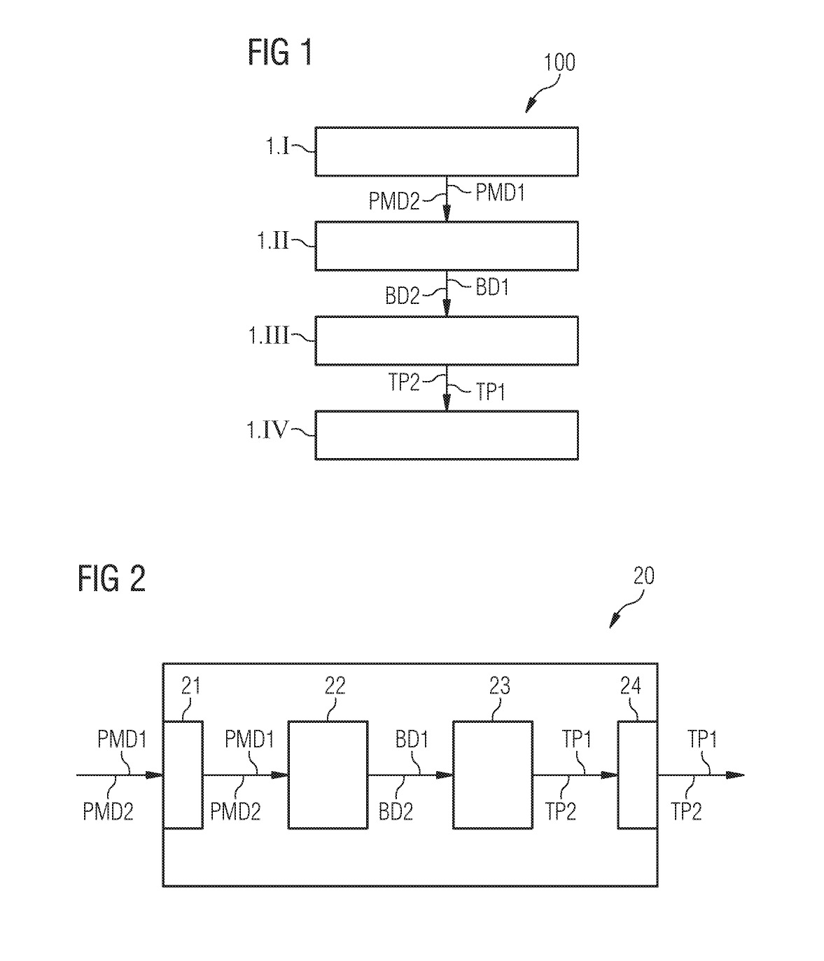

FIG. 1 shows a flow diagram 100 which illustrates a method for determining tissue properties in an examination region. In advance, i.e. before the start of the method, a patient is injected with a contrast medium which passes via the blood circulation to the examination region in the body of the patient. Subsequently, in step 1.I the examination region is irradiated with X-rays and two sets PMD1, PMD2 of projection measurement data assigned to different X-ray energy spectra, also known as spectral projection measurement data, are acquired from the examination region. During the acquisition of the spectral projection measurement data PMD1, PMD2 in the examination region, the previously injected contrast medium is present in the examination region. In step 1.II, image data BD1, BD2 is then reconstructed on the basis of the acquired projection measurement data PMD1, PMD2. In the example embodiment shown in FIG. 1, two pseudo-monoenergetic image data sets BD1, BD2 are reconstructed on the basis of the projection measurement data PMD1, PMD2. A first image BD1 is reconstructed as a contrast image, i.e. a pseudo-monoenergetic image is calculated on the basis of the projection measurement data, wherein the X-ray energy associated with the image lies above the K-edge of the previously injected contrast medium. A second pseudo-monoenergetic image BD2 is reconstructed as a non-contrast image at a correspondingly low X-ray energy, the value of which lies below the K-edge of the contrast medium used.

Subsequently, in step 1.III, texture parameters TP1, TP2 or texture parameter values are determined on the basis of the reconstructed image data BD1, BD2. Texture parameters can concern, for example, the mean image intensity or the evenness or homogeneity or the form of the texture of the images BD1, BD2. Herein, parameter values can be determined for different filter sizes from “fine” to “coarse”.

Then, in step 1.IV a comparison of the determined texture parameters TP1, TP2 with one another takes place. I.e. texture parameter values of the contrast image BD1 are compared with the texture parameter values of the non-contrasted image BD2. On the basis of this comparison, for example, a tumor can be better localized and can be more easily recognized on the basis, for example, of a treatment of necrotic regions that occur. Furthermore, correlations between the texture parameter values of the different images BD1, BD2 can be investigated. The texture parameters TP1, TP2 determined and the correlations can subsequently be used to estimate the aggressiveness of a tumor, to predict the response of a tumor to a treatment and to monitor the response of a tumor during the treatment.

FIG. 2 is a schematic representation of an image analysis apparatus 20 according to an example embodiment of the invention. The image analysis apparatus 20 comprises an input interface 21 for receiving two spectral contrast medium-enhanced acquired projection measurement data sets PMD1, PMD2 from an examination region of a patient. The image analysis apparatus 20 also has an image reconstruction unit 22 which is configured to reconstruct at least two sets of image data BD1, BD2 on the basis of the acquired spectral projection measurement data PMD1, PMD2. Also part of the image analysis apparatus 20 is an image analysis unit 23 which is configured to determine texture parameters TP1, TP2 on the basis of the reconstructed image data BD1, BD2. The texture parameters TP1, TP2 determined or the texture parameter values are communicated via an output interface 24 to other units, such as a data storage unit or an image display unit. The texture parameter values TP1, TP2 determined can also be communicated to a diagnosis apparatus (not shown) which automatically determines, on the basis of the determined texture parameter values TP1, TP2 and comparison values or reference values, the aggressiveness of a tumor or its response to a treatment.

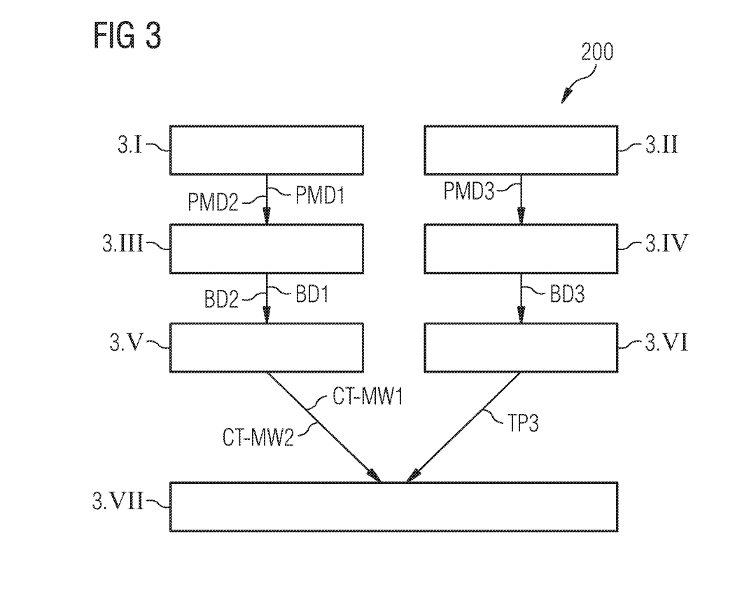

FIG. 3 shows a flow diagram which illustrates a method for determining tissue properties in an examination region according to an alternative example embodiment of the invention. In the step 3.I, initially as in the example embodiment illustrated in FIG. 1, a first and a second spectral projection measurement data set PMD1, PMD2 is acquired contrast medium-enhanced from an examination region. In addition, however, in contrast to the method illustrated in FIG. 1, in step 3.II, a third projection measurement data set PMD3 is acquired contrast medium-enhanced with the aid of a standard CT imaging method. On the basis of the first and second projection measurement data sets PMD1, PMD2, in step 3.III, a first and a second image data set BD1, BD2 are reconstructed and on the basis of the third projection measurement data set PMD3, in step 3.IV, a standard CT image BD3 is reconstructed. On the basis of the first and second image data sets BD1, BD2, as distinct from the example embodiment illustrated in FIG. 1, in step 3.V, first and second CT mean values CT-MW1, CT-MW2 are determined as parameter values. In addition, in step 3.VI, on the basis of the third image data set BD3, texture parameter values TP3 are determined.

With the aid of the mean values CT-MW1, CT-MW2 of the CT values and the texture parameter values TP3, in the step 3.VII, statements are made regarding the extent of an existing tumor and its aggressiveness and response to a treatment. As a result of the combination of the mean values CT-MW1, CT-MW2 of the CT values and the texture parameter values TP3, these estimates are made more precise and more reliable than estimates purely on the basis of texture parameter values.

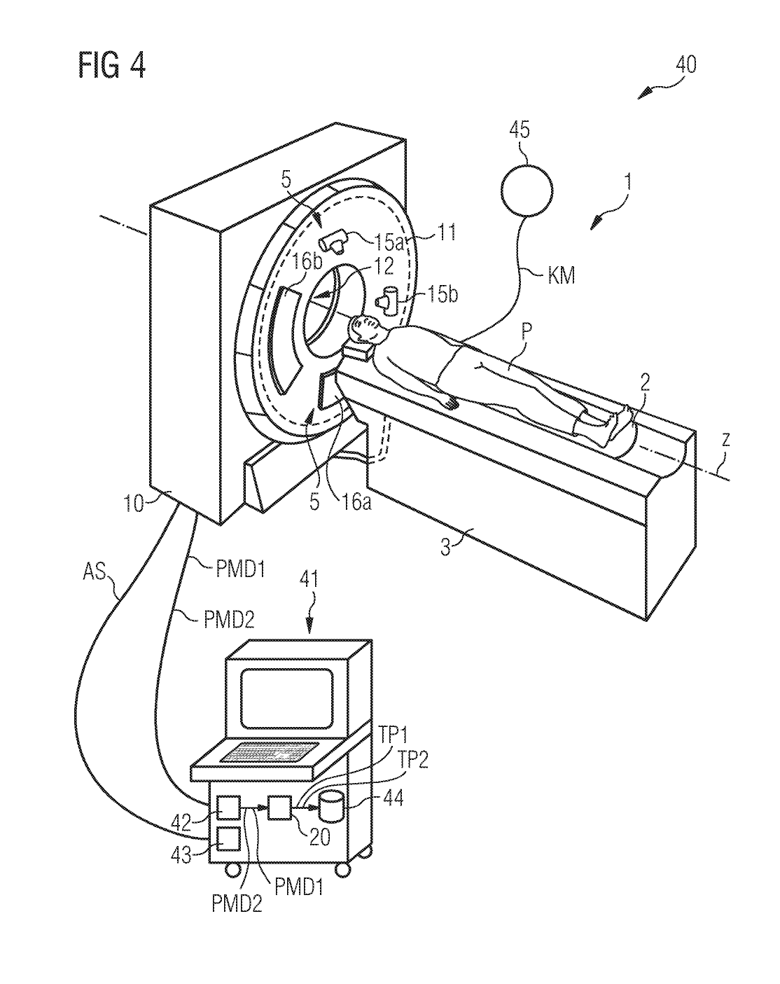

FIG. 4 shows a computed tomography system 40 which comprises the image analysis apparatus 20 shown in FIG. 2. The CT system 40 herein substantially consists of a typical scanning unit 10 in which a projection data acquisition unit 5 on a gantry 11 with two detectors 16a, 16b and X-ray sources 15a, 15b arranged respectively opposing the two detectors 16a, 16b circulates round a scanning space 12. Situated in front of the scanning unit 10 is a patient positioning apparatus 3 or a patient table 3, the upper part 2 of which can be displaced with a patient P situated thereon toward the scanning unit 10, in order to move the patient P through the scanning space 12 relative to the detectors 16a, 16b. The scanning unit 10 and the patient table 3 are controlled by a control device 41 from which acquisition control signals AS are transmitted via a conventional control interface 43 in order to control the whole system in the conventional manner according to pre-determined measurement protocols. In the case of a spiral acquisition, due to a movement of the patient P along the z-direction which corresponds to the system axis z through the scanning space 12 and the simultaneous circulation of the X-ray sources 15a, 15b for the X-ray sources 15a, 15b relative to the patient P during the scan, a helical path results. In parallel, herein each detector 16a, 16b always moves with and opposite to the respective X-ray source 15a, 15b in order to acquire projection measurement data PMD1, PMD2 which is then used for the reconstruction of dual-energy volume and/or slice image data. Similarly, a sequential scanning method can also be carried out in which a fixed position in the z-direction is approached and then, during a circulation, a partial circulation or a plurality of circulations at the z-position in question, the required projection measurement data PMD1, PMD2 is acquired, in order to reconstruct a sectional image at this z-position or to reconstruct image data from the projection data of a plurality of z-positions. The inventive method is also in principle usable with other CT systems, for example, with a detector forming a complete ring. For example, the inventive method can also be used on a system with an unmoved patient table and a gantry moved in the z-direction (a so-called sliding gantry).

In FIG. 4, a contrast medium injecting unit 45 is also shown which is configured to inject a contrast medium KM into a patient P in advance, i.e. before the start of a CT imaging method.

The projection measurement data PMD1, PMD2 (also known as raw data) acquired from the two detectors 16a, 16b is transferred via a raw data interface 42 to the control device 41. This projection measurement data PMD1, PMD2 is then further processed, possibly following a suitable pre-processing (e.g. filtration and/or radiation hardening correction), in an image analysis apparatus 20 according to at least one embodiment of the invention which in this example embodiment is realized in the control device 41 in the form of software on a processor. This image analysis apparatus 20 determines texture parameter values TP1, TP2 on the basis of the projection measurement data PMD1, PMD2.

The texture parameter values TP1, TP2 determined are subsequently transferred to an image data storage unit 44 from which they are transferred, for example, to an image display unit for pictorial display. By means of an interface (not shown in FIG. 4), they can also be fed into a network connected to the computed tomography system 40, for example, a radiological information system (RIS), and stored in a mass memory store accessible there or output to printers connected there. The data can thus be further processed in any desired manner and then stored or output.

The components of the image analysis apparatus 20 can be realized mainly or completely in the form of software elements on a suitable processor. In particular, the interfaces can also be configured purely as software between these components. It is required only that access possibilities exist in suitable memory regions in which the data can be suitably placed in intermediate storage and called up again and updated at any time.

Finally, it should again be noted that the medical technical devices and methods described above in detail are merely example embodiments which can be modified by a person skilled in the art in a wide variety of ways without departing from the scope of the invention. Furthermore, the use of the indefinite article “a” or “an” does not preclude the relevant features also being present plurally. It is also not precluded that elements of the present invention represented as individual units consist of a plurality of cooperating subcomponents which can also be spatially distributed, if necessary.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

what is claimed is:

1. A method for carrying out a parameter analysis in an examination region, the method comprising:
acquiring contrast medium-enhanced projection measurement data, including at least two spectral projection measurement data sets of the examination region;
reconstructing image data based on the acquired contrast medium-enhanced projection measurement data, wherein the reconstructed image data includes at least two spectral image data sets;
generating a parameter database, including establishment of texture parameters, based upon the reconstructed image data; and
carrying out the parameter analysis based upon the generated parameter database.

2. The method of claim 1, wherein the parameter analysis comprises analyzing of parameter correlations.

3. The method of claim 1, wherein the texture parameters are determined based upon the at least two spectral image data sets.

4. The method of claim 1, wherein in the generating of the parameter database, CT mean value parameters are determined based upon the at least two spectral image data sets.

5. The method of claim 1, wherein
based upon the contrast medium-enhanced projection measurement data, a standard image data set is reconstructed; and
the texture parameters are determined based upon the standard image data set.

6. The method of claim 5, wherein
in the acquiring of the contrast medium-enhanced projection measurement data, a standard projection measurement data set is also acquired, and
the standard image data set is reconstructed based upon the additional standard projection measurement data set.

7. The method of claim 5, wherein the standard image data set is obtained as a mixed image of a plurality of spectral image data sets.

8. The method of claim 1, wherein the at least two spectral image data sets include pseudo-monochromatic image data.

9. The method of claim 1, wherein the at least two spectral image data sets include one of the following image combinations:
an iodine image and a virtual non-contrast image, or
a series of monochromatic images.

10. The method of claim 1, wherein based upon the parameter analysis, one of the following items of information is determined:
a characterization of a tumor,
an expected response of a tumor to a particular treatment, or
an actual response of the tumor during a treatment.

11. An image analysis apparatus, comprising:
an input interface to receive contrast medium-enhanced projection measurement data including at least two spectral projection measurement data sets of an examination region of a patient;
an image reconstruction unit to reconstruct image data based upon the contrast medium-enhanced projection measurement data , wherein the reconstructed image data includes at least two spectral image data sets; and
an image analysis unit to generate a parameter database, comprising the establishment of texture parameters based upon the reconstructed image data and to carry out a parameter analysis based upon the generated parameter database.

12. A computed tomography system, comprising:
a scanning unit to acquire the contrast medium-enhanced projection measurement data from the examination region of the patient; and
the image analysis apparatus of claim 11.

13. A non-transitory computer program product, comprising a computer program, directly loadable into a storage apparatus of a computed tomography system, the computer program including program portions configured to carry out the method of claim 1 when the computer program is executed in the computed tomography system.

14. A non-transitory computer-readable medium including executable program portions stored thereon which are configured to be read in and executed by a computer unit to carry out the method of claim 1 when the program portions are executed by the computer unit.

15. The method of claim 2, wherein the texture parameters are determined based upon the at least two spectral image data sets.

16. The method of claim 2, wherein in the generating of the parameter database, CT mean value parameters are determined based upon the at least two spectral image data sets.

17. The method of claim 2, wherein
based upon the contrast medium-enhanced projection measurement data, a standard image data set is reconstructed; and
the texture parameters are determined based upon the standard image data set.

18. The method of claim 17, wherein
in the acquiring of the contrast medium-enhanced projection measurement data, a standard projection measurement data set is additionally acquired, and
the standard image data set is reconstructed based upon the additionally acquired standard projection measurement data set.

19. The method of claim 6, wherein the standard image data set is obtained as a mixed image of a plurality of spectral image data sets.

20. The method of claim 2, wherein the at least two spectral image data sets include pseudo-monochromatic image data.

21. The method of claim 2, wherein the at least two spectral image data sets include one of the following image combinations:
an iodine image and a virtual non-contrast image, or
a series of monochromatic images.
